(19) United States
(12) Patent Application Publication
(10) Pub. No.: US 2007/0264361 A1
(43) Pub. Date: Nov. 15, 2007

(54) Composition for Suppressing Cyclooxygenase and/or 5-Lypoxygenase

(75) Inventors: Tae-Hyung Jo, Gyeonggi-do (KR); Sung-Sick Woo, Seoul (KR); Ji-Min Cha, Seoul (KR); Dong-Seon Kim, Daejeon (KR); Young-Chul Lee, Daejeon (KR); Seon-Gil Do, Chungcheongbuk-do (KR); Jong-Han Kim, Gyeongsangnam-do (KR); Tae-Woo Kim, Ulsan (KR); Hee-Sun Sung, Gyeonggi-do (KR)

Correspondence Address: BIRCH STEWART KOLASCH & BIRCH, PO BOX 747, FALLS CHURCH, VA, 22040-0747, US

(73) Assignee: UNIGEN, INC., CHUNGCHEONGNAMDO (KR)

(21) Appl. No.: 11/661,382

(22) PCT Filed: Sep. 1, 2005
(86) PCT No.: PCT/KR05/02888

§ 371 Date: Apr. 4, 2007

(30) Foreign Application Priority Data
Sep. 1, 2004 (KR) 10-2004-0069609

(51) Int. Cl. A61K 36/51; A61K 36/539; A61K 36/82; A61P 29/00

(52) U.S. Cl. 424/729; 424/745; 424/774

(57) ABSTRACT

The present invention relates to a composition for the prevention or treatment of physiological and pathological disorders mediated by cyclooxygenase (COX) and/or 5-lipoxygenase (5-LO) comprising Uncaria genus plant, in particular, Uncaria gambir, or its extract, and to a combined composition of said Uncaria genus plant extract and Scutellaria baicalensis extract and/or Camellia sinensis extract. The present composition shows excellent COX and/5-LO inhibition effects, and thus can be used for the prevention or treatment of disease and disorders mediated by various COX pathway and/or 5-LO pathway, including osteoarthritis and rheumatoid arthritis.

### TECHNICAL FIELD

The present invention relates to a composition for the prevention or treatment of physiological and pathological disorders mediated by cyclooxygenase (‘COX,’ below) and/or 5-lipoxygenase (‘5-LO,’ below) comprising Uncaria genus plant or its extract, specifically, Uncaria genus plant alone and additionally including Scutellaria baicalensis and/or Camellia sinensis extract.

### BACKGROUND ART

Improvement of the living standard and change of the living style by the socio-economic development, and increase of the average life span have brought great change in the aspect of disease according to increase of the aged population, and chronic diseases have gained more weight than epidemic diseases in the cause of death. Chronic diseases now draw more socio-economic attention in terms of increase of medical expenses, increase of required medical standard, search of ways to decrease them and the like. Most representative chronic diseases include arthritis, decrease of cognitive function, dermatitis, gastritis, hypertension, diabetes, paradentitis, etc.

Arthritis is the most important cause to restrain daily life activities of a human being, and the outbreak frequency is particularly high in female and old people. Arthritis can be divided into Osteoarthritis (degenerative arthritis) and Rheumatoid arthritis.

Osteoarthritis is caused by degeneration of body joints, accompanying pain and inflammation from wear or damage of joint cartilage of conjugated regions between bones (buttocks, knee, neck, waist, finger, toe knuckle, etc.). Normally, joint cartilage is destroyed and regenerated, but if the amount of destroyed cartilage is more than that of regenerated cartilage, the amount of joint cartilage to absorb impact is decreased or worn out. Then, the bones between joints come in contact with each other, followed by extreme pain. Such damage of joint cartilage is the beginning of osteoarthritis, and extreme pain is caused if no treatment is done.

Rheumatoid arthritis is an inflammatory autoimmune disease occurred in multiple ways in many joints. In case of arthritis patient, at the same time as the synovial membrane tissue of a joint becomes hyperplasia, macrophage, dendritic cell, and activated T lymphocyte and B lymphocyte move into the synovial membrane tissue, and polymorphonuclear cell is accumulated in the synovial fluid and on the surface of cartilage to induce inflammation. Such inflammation of synovial membrane tissue is inferred to be induced by reaction of T lymphocyte with self-antigen which is not identified yet. In this reaction, T lymphocyte infiltrated into most tissues does not show activation mark on the cell surface, and cytokine is hardly expressed, either. However, a large amount of cytokine originated from macrophage is observed in synovial membrane tissue and synovial fluid with rheumatoid arthritis symptoms. Representative cytokine includes interleukin-1 (IL-1) and tumor necrosis factor-α (TNF-α) which are known to stimulate growth of synovial membrane fibroblast. These experimental results support a theory that T lymphocyte plays a very important role in inducing inflammation of synovial membrane tissue, and inflammation symptom thereafter is maintained by cytokine originated from activated synovial membrane cells [Carson D. A. et al., J. Clin. Invest., 87, pp 379-383, 1991: Tighe H. et al., J. Exp. Med., 177, pp 10-118, 1993: Bumiester G. R. et al., Arthritis Rheum, 40, pp 5-18, 1997: Panayi G. S., Curr. Opin. Rheumatol., 9, pp 236-240, 1997].

Anodyne or antiphlogistic agent is generally used in order to alleviate pain including arthritis, and a representative drug is NSAIDs (Nonsteroidal Anti-Inflammatory Drugs) having COX inhibiting effect [UK-1, R Braham, B Dawson, C Goodman, The effect of glucosamine supplementation on people experiencing regular knee pain, Br. J. Sports. Med. 2003; 37:45-49]. NSAIDs such as aspirin are the best selling prescription drug, and are used for the treatment of degenerative arthritis, rheumatoid arthritis, headache since they are effective for anti-inflammation, alleviation of fever, and alleviation of pain. In case where these NASIDs are used for arthritis, they slightly improve the symptom, but do not stop the cartilage loss in the joint region nor the progress of the disease. In addition, they have serious side effects as gastroenteric trouble, and thus about a half of the patients who took NASIDs stop taking them within one year. Thus, there has been a need for a new therapeutic agent. An agent selectively inhibiting COX-2 or a therapeutic agent simultaneously inhibiting COX-2 and 5-LO has been developed.

Inflammatory reaction is caused when isolation and metabolism of arachidonic acid from cellular membrane produce pro-inflammatory metabolite in many pathways. The two important pathways to inflammation, COX-2 and 5-LO, are enzymes to play an important role in the arachidonic acid (AA) cascade, and these pathways are occurred in parallel with the pathways producing leukotrienes and prostaglandine which play an important role in initiating and progressing inflammatory reaction, respectively. COX is an enzyme which reacts as catalyst in the conversion process of arachidonic acid into prostaglandines (‘PGs’, below) after the conversion of phospholipid of cellular membrane into arachidonic acid. Such produced PGs may stimulate smooth muscle contraction depending on their kinds, and decrease or increase blood pressure or blood cohesion depending on animals. In addition, they play a role to accelerate ion transport in membrane, stimulate inflammation, and prevent lipid degradation in lipid tissue. Therefore, an enzyme to be the cause of production of these inflammatory mediators has been a target for many new drugs aiming at the treatment of inflammation which is the cause of rheumatoid arthritis, osteoarthritis, dermatitis, cognitive function related disease and cancer, or degenerative disease.

Two kinds of COX, COX-1 and COX-2, are known in the art. COX-1 is consistently expressed in most tissues, and plays a role of “house keeping.” That is, it participates in production of PG which is present in gastric mucous membrane and expands blood vessels to maintain kidney function, and production of blood platelet thromboxane.

COX-2 is not expressed in most normal tissues, and induced by previously expressed growth factors under disease or physiological condition. In particular, it is known to be widely induced by cytokine causing pro-inflammation.

NSAIDs, which have been used for the treatment of inflammation until now, inhibit even COX-1 which is consistently expressed in normal tissues, and so have caused side effects such as gastric mucous membrane corrosion, ulcer, etc. Recently, COX-2 selective inhibiting agents have been developed as a new agent improving this. CeleCOXib is a representative COX-2 selective inhibiting agent which is now clinically used as anti-inflammatory agent and anti-cancer agent. It is effective for the treatment of osteoarthritis and rheumatoid arthritis, and the decrease of the number of polyp present in the colon of patient with familial adenomatous polyposis (FAP).

Another enzyme which participates in the metabolism process of arachidonic acid into chemical transmitter in inflammatory reaction is lipoxygenase. There are three kinds of lipoxygense, 5-, 12-, and 15-lipoxygenase, among which 5-lipoxygenase participates in the synthesis process of leukotriene A₄, B₄, C₄, D₄, E₄ (LTA₄, LTB₄, LTC₄, LTD₄, LTE₄), etc. from arachidonic acid via 5-HPETE. Samuelsson et al. disclose that among these leukotrienes, LTB₄ is one of leukocytes acting in the second stage of inflammatory reaction, and is biosynthesized mainly in polymorphonuclear leukocyte (PMNL) and known as showing functions such as leukocyte cohesion, infiltration, isolation of chemotaxis and lysosomal enzyme, etc. And, many scientists have conducted researches for factors related to 5-LO activation and development of drugs for inhibiting such activation, but the result has been insignificant and only ETYA and BW_{755C} have been developed as drugs [Kyung-rak MIN et al., Activation of 5-lipoxygenase and leukotriene B₄ biosynthesis inhibiting material, Pharmacology, Vol. 33(6), 319-323 (1989)].

The reaction mechanism of COX inhibitor is identical to that of most conventional NSAIDs, and thus COX inhibitor is used for the treatment of many conditions such as pain and swelling caused by inflammation in temporary disorders and chronic diseases wherein inflammation plays an important role. Temporary disorders refer to slight abrasion, sunburn, contagious dermatitis, headache, menstrual pain, etc. Chronic diseases refer to decrease of cognitive function, rheumatoid arthritis, osteoarthritis, etc.

COX inhibitor is also used in skin disorders like skin scleroma as well as systemic lupus erhthromatosus (SLE) [Goebel et al., Chem. Res. Tox., 12:488-500, 1999, Patrono et al., J. Clin. Invest., 76:1011-1018, 1985]. In addition, COX inhibitor is also used for the alleviation of inflammatory, not rheumatoid, skin disorder such as psoriasis, wherein it shows direct effect by decreasing inflammation from overproduction of prostaglandine [Fogh et al., Acta Derm Venerologica, 73:191-193, 1993].

COX inhibitor plays a potential role for cancer treatment in addition to its use for anti-inflammatory drugs. Over-expression of COX has been observed in many human malignant tumors, and COX inhibitor shows effective for the treatment of animals suffering from cutaneous cancer, breast cancer and bladder cancer. Although the reaction mechanism is not completely identified, over-expression of COX has shown to inhibit cell death and increase an invasion of tumorigenic cell type [Dempke et al., J. Can. Res. Clin. Oncol., 127:411-417, 2001, Moore and Simmons, Current Med. Chem., 7:1131-44, 2000].

In addition, an interrelation between COX expression, general inflammation and pathogenesis of Alzheimer's disease has been confirmed due to recent scientific improvement [Ho et al., Arch. Neurol., 58:487-92, 2001]. In animal model, a genetically transformed mouse over-expressing COX enzyme has much more vulnerable neuron. NIA (National Institute on Aging) started a clinical test to confirm whether or not NSAID can delay the progress of Alzheimer's disease, and many reports show that the inhibition of COX generated in inflammatory reaction helps cognitive function improvement [Cemak I., Exp Brain Res., 147(2):193-9, 2002, Casolini P., J Neurosci Res., 68(3):337-43, 2002, Andreasson K I., J Neurosci., 21(20):8198-209, 2001]. In addition, it was confirmed that COX inhibitors are effective for mental disorder [Muller N., Expert Opin Investig Drugs, 13(8):1033-44, 2004].

Also, there is a report that suppressors inhibiting both COX-2 and 5-LO inhibit arterial tube contraction in aged heart of a mouse model [Gok et al., Pharmacology, 60:4146, 2000].

Uncaria gambir is a plant which belongs to madder family. It grows naturally in all over the East Indies, and is cultivated in Malaysia, China, India, Sumatra, and Brunei. A white flower blossoms at the axilla of leaf. When the flower is fallen, the flower stalk bends hooked to wind other plant. One year after sowing Uncaria gambir seeds, so-called water extract can be obtained from cutting and extracting an end part of the leaf stem every 4-8 months. This water extract can be obtained most from the 6-year-old tree. When the tree grows for about 15 years, the farm should be plowed to separate the roots. This water extract contains d- and dl-catechin (catechol), tannic acid, quercetin, and alkaloid gambirin.

The extract of Uncaria gambir is used for medicinal purposes, and also largely used for brown dyes or leather tanning. In particular, some peoples in Southeast Asia eat Uncaria gambir mixed with water by pasting the mixture on Bin-ran-za. The water extract as astringent is widely used for making chewing drugs such as In-dan. According to Dong-Eui Treatment, the water extract was used as astringent or blood coagulating agent for wound, sores in mouth, bloody excrement, bloody urine, hemoptysis, leucorrhea, and other dermatosis [Korea Food and Drug Administration]. In addition, Dong-Eui-Bo-Gam teaches that Uncaria gambir can be used for the treatment of pain from the swollen tendon and bone as “saengbomyungdan, yangmaechang, chunpochang, whanchang, and gyungbundok.”

Scutellariae Radix refers to the root of Scutellaria baicalensis, a perennial herb, which belongs to Labiatae genus. This plant is perennial and blossoms in July to September after 2 years. The stem grows straight and thick, but in fertile soil, it grows slantingly or even lies down. The height of stem is within 40˜60 cm, the leaf is symmetrical and of the form of lightning rod without leafstalk. The flower is raceme, and gathers and blossoms at the end of branch, and the shape of flower is labiate and open. The root is collected in autumn or spring 3 to 4 years after planting, and after removing the periderm, it is air-dried and used for medicinal purposes. Then, the roots' color is yellow. Generally, both xylem and parenchymal of this medicinal herb are bulky, and thus mostly the pith is empty and so popularly called as grass of rotten pith. However, in Japan, its fresh roots having a filled pith are called as Cha-geum, ones having an empty pith as Sook-geum, crushed ones as Pyan-geum, and the like. In Korea, they are also called as Ko-Geum, Won-geum, Kyung-geum, Kong-jang, and the like.

Scutellaria baicalensis, a Chinese medicinal plant, contains a great deal of free-B-ring flavonoid including baicalein, baicalin, wogonin, and baicalenoside. Traditionally, Scutellaria baicalensis was used for the treatment of disorders including clearing away, purging fire, dampness-warm, summer fever syndromes, polydipsia, carbuncle, scarlet fever, dysentery, hematemesis and epistaxis. In addition, it was used for the prevention of miscarriage. Scutellaria is now clinically used for the treatment of disorders including pediatric bacterial diarrhea, hypertension, bronchial asthma and upper respiratory infections. A report shows that the pharmacological effect of Scutellaria's roots for the treatment of bronchial asthma is associated with the presence of free-B-ring flavonoid and the inhibition of eotaxin related to eosinophil infiltration [Nakajima et al., (2001) Planta Med. 67(2):132-135].

Camellia sinensis, which recently draws attention as health food, contains many useful components. Among the components, catechin compound has relatively high anti-oxidation effect, and so many researches thereon are in progress. Catechin compound in Camellia sinensis includes epigallocatechin (EGC), epicatechin (EC), epigallocatechin gallate (EGCG), epicatechin gallate (ECG), etc. In addition to the excellent anti-oxidation effect, the catechin compound has such effects as anti-cancer effect, immune system reinforcement, cutaneous cancer prevention, anti-thrombus effect, heart disease prevention, cholesterol prevention, etc. Therefore, consistent researches have been carried out for this catechin compound in Camellia sinensis in the beverage and pharmaceutical field. The researches have been most actively carried out in China, and many products for Camellia sinensis are now commercialized there. Simingshan natural biological product Co., Ltd., China tianbao biochemical plant, and HealthLand Supplies Ltd., etc. are the leading companies that have carried out sales and research of Camellia sinensis extract product. In Japan, as a result of research on catechin compound, ‘β-catechin’ by Daedong pharmaceuticals Co., Ltd. and ‘catechin compound powder’ by Samjung agriculture and forestry Co., Ltd. have been commercialized, and consistent research and investment have been poured to develop higher yield and economic process.

Another effective component of Camellia sinensis, polyphenol flavones, inhibits growth of colonocytes which becomes cancerous by a certain amount of mRNA for COX-2, NFκB (Nuclear Factor kappa B), and bcl-X(L) genes. As can be seen from the basic structure illustrated below, free-B-ring flavones and free-B-ring flavonols are specific kind of flavonoid compound which substituent group does not exist in B-ring structure among aromatic compounds [Korean Patent Laid-open Publication No. 10-2004-0025884].

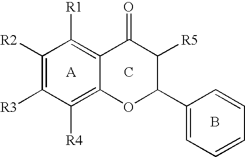

There has been no report showing that Uncaria genus plant including Uncaria gambir can be used as anti-inflammatory drugs. In particular, it was not known that a combination of Uncaria gambir and Scutellaria baicalensis and/or Camellia sinensis can be used as anti-inflammatory drugs, specifically for the prevention or treatment of disease and disorders mediated by COX pathway and/or 5-LO pathway, including osteoarthritis or rheumatoid arthritis.

### DISCLOSURE OF THE INVENTION

Distinguishably from the development strategy in Western advanced countries, the present inventors have continued to search natural products to develop new COX and/or 5-LO inhibiting drugs. As a result, they discovered that Uncaria genus plant including Uncaria gambir has COX and/or 5-LO inhibition effects. Also, to find out another natural medicine showing synergistic effect with said extract, they have conducted many experiments using in vitro test (COX-1 and 2,5-LO) and in vivo test [Swelling, CIA (Collagenase Induced Arthritis) model], and GAG analysis to confirm joint protection effects. As a result, they additionally discovered that a mixture of combining Scutellaria baicalensis extract and/or Camellia sinensis extract shows much improved synergistic effect on COX and/or 5-LO inhibition activity, and measured the synergistic effect by using COLBY formula (COLBY S. R., Calculating synergistic and antagonistic response of herbicide combinations, Weeds 15, 20-22, 1967), to complete the present invention.

Thus, an object of the present invention is to provide a composition for the prevention or treatment of physiological and pathological disorders mediated by COX and/or 5-LO comprising a new plant extract showing COX and/or 5-LO inhibition effects, namely, Uncaria genus plant, or additionally comprising Scutellaria baicalensis extract and/or Camellia sinensis extract.

Another object of the present invention is to provide a composition for the prevention or treatment of physiological and pathological disorders mediated by COX and/or 5-LO comprising Scutellaria baicalensis extract and Camellia sinensis extract.

Another object of the present invention is to provide a use of the above composition to prevent or treat physiological and pathological disorders mediated by COX and/or 5-LO.

Another object of the present invention is to provide a method for preventing or treating physiological and pathological disorders mediated by COX and/or 5-LO by administering a therapeutically effective amount of the above composition to mammal.

Another object of the present invention is to provide a method of preparing an agent for the prevention or treatment of physiological and pathological disorders mediated by COX and/or 5-LO, by mixing Uncaria genus plant with Scutellaria baicalensis extract and/or Camellia sinensis extract, or mixing Scutellaria baicalensis extract with Camellia sinensis extract.

### BRIEF DESCRIPTION OF THE DRAWINGS

FIG. 1 is a graph showing the anti-inflammatory activities of Examples 1 and 2.

FIG. 2 is a graph showing the anti-inflammatory activities of Examples 5 to 8.

FIG. 3 is a graph showing the anti-inflammatory activities of Example 5 and Examples 1 and 3.

FIG. 4 is a graph showing the anti-inflammatory activities of Example 6 and Examples 1 and 4.

FIG. 5 is a graph showing the anti-inflammatory activity of Example 7.

FIG. 6 is a graph showing the anti-inflammatory activities of Example 8 and Examples 3 and 4.

FIG. 7 is a graph showing the change of swelling of the mouse paw by time after administering Example 5.

FIG. 8 is a graph showing the change of arthritis index by time after administering Example 5.

FIG. 9 is a photograph showing the cartilage tissue of CIA mouse joint after administering Example 5.

FIG. 10 is a graph showing the joint protection effects of Examples 1 to 4.

FIG. 11 is a graph showing the joint protection effects of Examples 5 to 8.

### BEST MODE FOR CARRYING OUT THE INVENTION

To achieve the above objects, the present invention provides a composition for the prevention or treatment of physiological and pathological disorders mediated by COX and/or 5-LO comprising Uncaria genus plant or its extract.

The present invention also provides a composition for the prevention or treatment of physiological and pathological disorders mediated by COX and/or 5-LO comprising said Uncaria genus plant and additionally Scutellaria baicalensis extract and/or Camellia sinensis extract.

The present invention also provides a composition for the prevention or treatment of physiological and pathological disorders mediated by COX and/or 5-LO comprising Scutellaria baicalensis extract and Camellia sinensis extract.

The present invention also provides a use of a composition comprising Uncaria genus plant or its extract; a composition comprising Scutellaria baicalensis extract and Camellia sinensis extract; and a composition comprising Uncaria genus plant or its extract and Scutellaria baicalensis extract and/or Camellia sinensis extract, to prevent or treat physiological and pathological disorders mediated by COX and/or 5-LO.

The present invention also provides a method for preventing or treating physiological and pathological disorders mediated by COX and/or 5-LO by administering a therapeutically effective amount of a composition comprising Uncaria genus plant or its extract; or a composition comprising Scutellaria baicalensis extract and Camellia sinensis extract, to mammal.

The present invention also provides a method of preparing an agent for the prevention or treatment of physiological and pathological disorders mediated by COX and/or 5-LO, by mixing Uncaria genus plant or its extract with Scutellaria baicalensis extract and/or Camellia sinensis extract in the weight ratio of 0.1˜10:0.1˜10, or mixing Scutellaria baicalensis extract with Camellia sinensis extract in the weight ratio of 0.1˜10:0.1˜10.

It is preferable to select one or more Uncaria genus plants from the group consisting of Uncaria gambir, U. attenuata Korth., U. borneensis Havil., U. callophylla Korth., U. elliptica R. Br., U. guianensis (Aubl.) Gmel., U. homomalla Miq., U. lanosa var. glabrata (B1.) Ridsd., U. macrophylla Wall., U. rhynchophylla Miq., U. sinensis (Oliv.) Havil., U. tomentosa (Willd.) DC., U. yunnanensis Hsia K. C., U. hirsuta Havil., and U. lanosa var. appendiculata f. setiloba (Benth.) Ridsd., and particularly preferable to use Uncaria gambir.

In the present composition, Uncaria genus plant, Scutellaria baicalensis, and Camellia sinensis can be used by commercially purchasable conventional herb material, and also can be used by a whole herb, branch, shell, leaf, sprout, root, endodermis, etc., preferably used in the form of powder or extract.

The Uncaria genus plant, Scutellaria baicalensis, and Camellia sinensis extract of the present invention can be used by extracting Uncaria genus plant, Scutellaria baicalensis and Camellia sinensis with water, organic solvent, or mixing solvent thereof. All conventional solvents can be used as the above organic solvent, preferably polar solvent such as water, C₁₋₄ alcohol, etc., or non-polar solvent such as n-hexane, dichloromethane, etc., or mixing solvent thereof.

The non-polar solvent extract of the present invention comprises extract extracted with non-polar solvent selected from the group consisting of n-hexane, dichloromethane, chloroform, or ethylacetate, preferably n-hexane, dichloromethane, and ethylacetate. In addition, the polar solvent extract of the present invention comprises extract extracted with polar solvent selected from acetone, water, C₁₋₄ alcohol such as methanol, ethanol, propanol, butanol, etc., or isopropyl alcohol.

The above extraction may be carried out by conventional methods such as hot water extraction, sonication, etc., and a lyophilized product of the extract can be used for the present composition.

In addition, the extract can be further purified by conventional fractionation method or chromatography, and such fractionated material or purified material is also within the scope of the present invention.

The composition of the present invention shows excellent COX and/or 5-LO inhibition effects, and can be used for the prevention or treatment of disease and disorders mediated by various COX pathway and/or 5-LO pathway, particularly including osteoarthritis and rheumatoid arthritis, without any side effects by using natural herb medicine.

In the present specification, the term, “physiological and pathological disorders mediated by COX and/or 5-LO pathway,” includes, for example, disease and disorders selected from the group consisting of inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer.

In the composition of the present invention, Uncaria genus plant, in particular, Uncaria gambir, can be used alone, but it is preferable to use a combined composition that Uncaria genus plant or its extract is additionally mixed with Scutellaria baicalensis extract, Camellia sinensis extract, or Scutellaria baicalensis and Camellia sinensis extract to show synergistic effect.

In particular, as shown in the following experimental example, Scutellaria baicalensis extract alone did not show COX and/or 5-LO inhibition effects. However, surprisingly, when Uncaria genus plant, particularly Uncaria gambir extract, was administered in combination with Scutellaria baicalensis and/or Camellia sinensis extract, and when a combination of Scutellaria baicalensis and Camellia sinensis extract was administered, a synergistic effect was observed.

In the composition of the present invention, the synergistic effect at the time of administering the combination in comparison with administration of the extract alone was measured and confirmed by using COLBY formula (COLBY S. R., Calculating synergistic and antagonistic response of herbicide combinations, Weeds 15, 20-22, 1967).

As shown above, when Uncaria genus plant, particularly Uncaria gambir, is used in combination with Scutellaria baicalensis and/or Camellia sinensis extract, their weight ratios of Uncaria gambir:Scutellaria baicalensis:Camellia sinensis could be in 0.1˜10:0.1˜10:0.1˜10, preferably 1˜10:1˜10:1˜10, preferably 1˜7:1˜7:1˜7. And, when Scutellaria baicalensis and Camellia sinensis are combined, they can be mixed in the weight ratio of 0.1˜10:0.1˜10, preferably 1˜10:1˜10, more preferably 1˜7:1˜7.

The composition of the present invention can be prepared into conventional pharmaceutical preparations according to conventional methods in the pharmaceutical field, for example, solution such as drinks, syrup, capsule, granule, tablet, powder, pill, ointment, and emulsion, skin external preparation such as gel, etc., by mixing it with a pharmaceutically acceptable carrier, excipient, etc.; and can be administered orally or parenterally. Preferably, the composition of the present invention may be orally administered in capsule, tablet and drink before and/or after the meal for quick effect.

Capsule, tablet, powder, granule, solution, pill, etc. comprising the composition of the present invention are preferably used as medicine or health care products. In this invention, “health care products” mean food products prepared and processed in the form of tablet, capsule, powder, granule, solution, pill, etc., by using material or ingredients having useful function to the human body.

The composition of the present invention is appropriately administered depending on the extent of absorption of active ingredients into the body; excretion rate; age, weight, sex, and condition of patient; severity of treated disease, etc. However, generally, it is preferable to administer the present composition in solution to adult by 0.01˜500 mg/kg, preferably 0.1˜200 mg/kg, per day, 1˜3 times a day. In other preparations, an appropriate amount based on the above dose for solution can be administered orally.

Hereinafter, the present invention will be described in more detail with reference to the following examples, but the scope of the present invention should not be construed to be limited thereto in any manner.

### EXAMPLES

1) Preparation of Uncaria gambirs, Scutellaria baicalensis and Camellia sinensis Extracts

### Example 1

### Preparation of Uncaria gambir Hot Water Extract

Young leaves of Uncaria gambir (50g) were steamed with hot steam. Then, they were extracted by adding purified water and squeezed out the juice, and the collected juice solution was slowly cooled and recrystallized, to give 7.87 g of Uncaria gambir extract powder (yield: 15.74%).

### Example 2

### Preparation of Uncaria gambir Ethanol Extract

Young leaves of Uncaria gambir (50 g) were extracted by adding ethanol and squeezed out the juice, and the collected juice solution was slowly cooled and recrystallized, to give 7.65 g of Uncaria gambir extract powder (yield: 15.3%).

### Example 3

### Preparation of Scutellaria baicalensis Extract

Scutellaria baicalensis (50 g) was put in 1L round-bottom flask, and by adding purified water (350 ml), extracted under reflux at 80° C. for 2 hr. The extract was cooled, filtrated and concentrated, to give 16.5 g of Scutellaria baicalensis extract powder (yield: 32.95%).

### Example 4

### Preparation of Camellia sinensis Extract

Camellia sinensis (50 g) was put in 1L round-bottom flask, and by adding aqueous ethanol (500 ml), extracted under reflux at 85° C. for 3 hr. The extract was cooled, filtrated and concentrated, to give 13.5 g of Camellia sinensis extract powder (yield: 27%).

### Example 9

### Extraction of Other Uncaria Genus Plant

Uncaria sinensis (Olv.) Havil. (50 g) was put in 1L round-bottom flask, and by adding purified water (500 ml), extracted under reflux for 5 hr. The extract was cooled, filtrated and concentrated, to give 7 g of Uncaria sinensis extract powder (yield: 14%).

2) Preparation of Mixture

### Example 5

### Preparation of Mixture of Uncaria gambir and Scutellaria baicalensis

The mixture of Uncaria gambir and Scutellaria baicalensis was prepared as follows.

| Ingredient | Input amount (g) | Input ratio (%) |
|---|---|---|
| Example 1 | 2.6 | 10.20 |
| Example 3 | 18.4 | 72.16 |
| Maltodextrin | 4.5 | 17.65 |
| Total | 25.5 | 100.0 |

### Example 6

### Preparation of Mixture of Uncaria gambir and Camellia sinensis

The mixture of Uncaria gambir and Camellia sinensis was prepared as follows.

| Ingredient | Input amount (g) | Input ratio (%) |
|---|---|---|
| Example 1 | 2.60 | 10.20 |
| Example 4 | 15.30 | 60.0 |
| Maltodextrin | 7.60 | 29.8 |
| Total | 25.50 | 100.00 |

### Example 7

### Preparation of Mixture of Uncaria gambir, Scutellaria baicalensis and Camellia sinensis

The mixture of Uncaria gambir, Scutellaria baicalensis and Camellia sinensis was prepared as follows.

| Ingredient | Input amount (g) | Input ratio (%) |
|---|---|---|
| Example 1 | 2.60 | 10.20 |
| Example 3 | 18.40 | 72.16 |
| Example 4 | 2.60 | 10.20 |
| Maltodextrin | 1.90 | 7.45 |
| Total | 25.50 | 100 |

### Example 8

### Preparation of Mixture of Scutellaria baicalensis and Camellia sinensis

The mixture of Scutellaria baicalensis and Camellia sinensis was prepared as follows.

| Ingredient | Input amount (g) | Input ratio (%) |
|---|---|---|
| Example 3 | 18.4 | 72.16 |
| Example 4 | 2.6 | 10.20 |
| Maltodextrin | 4.5 | 17.65 |
| Total | 25.5 | 100.0 |

### Experimental Example

1) COX and/or 5-LO Inhibition Activity Test

(1) COX Inhibition Activity Test

\textcircled{1} Test Materials:

Materials: COX analysis kit (Cayman, Cat#760111), Indomethacin (Cayman, Cat#70270), AA-861 (Biomol, Cat#EI-216), H₂O₂ (Aldrich, Cat#216763)

Sample: Examples 1 to 8

Concentration: 10, 50, 500, 1000 μg/ml

\textcircled{2} Test Methods: Analysis Buffer (160 μl) and heme (10 μl) were put into Background Wells. Analysis Buffer (150 μl), heme (10 μl), and Enzyme (COX-1 or COX-2, 10 μl) were put into 100% of Initial Activity Wells. Analysis Buffer (150 μl), heme (10 μl), and Enzyme (COX-1 or COX-2, 10 μl) were put into Inhibitor Wells. The Sample (10 μl) dissolved in DMSO was put into the Inhibitor Wells. Instead of the Sample, DMSO (10 μl), the solvent which was used to dissolve the Sample, was put into 100% of Initial Activity Wells and Background Wells. After slow shaking, they were reacted at 25° C. for 5 min. 20 μl of colorimetric substrate was put into every well. And, 20 μl of arachidonic acid was put into every well (Final Conc. 100 μM). After slow shaking, they were reacted at 25° C. for 5 min. The reaction was stopped, the absorbance at 590 nm (590˜611 nm) was measured, and the relative activity of Test groups compared with Control group was calculated in % by the following Calculation formula.

Calculation formula:
% of Ihibition={(100%-inhibition)/(100%-blank)}×100

\textcircled{3} Test result: 50% Ihibition activity against COX-1,2 of single extract and mixture (Unit: μg/ml)

| | COX-1 | COX-2 |
|---|---|---|
| Example 1 | <10 | 25 |
| Example 2 | 13 | 22 |
| Example 3 | >1000 | 730 |
| Example 5 | 260 | 260 |
| Example 6 | 15 | 26 |
| Example 7 | 150 | 175 |
| Example 8 | 200 | 220 |

\textcircled{4} Conclusion: Among single extracts, Uncaria gambir extract showed excellent COX inhibition activity from the result of Example 1=Example 2>>Example 3. And, mixed compositions wherein said single extracts were mixed in proper ratios showed COX inhibition activity in the order of Example 6>Example 7>Example 8>Example 5.

As known from the above test, Uncaria gambir crude extract (Examples 1 and 2) showed excellent COX inhibition activity, whereas 5-LO inhibition activity of Scutellaria baicalensis extract was not significant. However, surprisingly, the mixture of said extracts showed synergistic COX inhibition activity.

(2) 5-LO (LTB₄ production inhibition)

\textcircled{1} Test Materials:

RPMI1640 medium: sigma Cet#R8758

T75 flask: Corning (430641)

Antibiotics: Gibco (15240-062)

FBS: biowhittaker (14-471QM)

Micro tube: sarstedt (72.690)

PBS: biowhittaker (17-512F)

Centrifuge: Hanil (micro-12)

Sample: Examples 1 to 8

Concentration: 0.025, 0.05, 1, 20 μg/ml (Examples 1 to 3) 0.005, 0.05, 0.5, 5 μg/ml (Examples 4 to 7)

\textcircled{2} Test Methods: HT-29 cell line (Korea Cell Line Bank) was cultured in T75 flask under the conditions of 5% CO₂ and 37° C. in 20 mL of RPMI1640 medium (10% of FBS), and was passaged 2-3 times a week. HT-29 cell line was seeded into 6-well plate in 1.5-2.0×10⁵/well/2 ml, and was cultured in the conditions of 5% CO₂ and 37° C. until it shows about 60-70% of confluence. After removing the medium, the cell was washed 2-3 times by PBS (biowhittaker, 17-512F), and 2mL of new medium (5% FBS, biowhittaker, 14-471QM) was added thereto. The Sample was treated to make the last concentration 0, 0.005, 0.05, 0.5, and 5 μg/ml. In addition, LPS was treated to make the last concentration 1 μg/ml. Non-treated N-Control was treated with the solvent which was used to dissolve the Sample (less than 0.1% DMSO), instead of the Sample. P-Control was treated with LPS only. Every Sample was reacted in the conditions of 5% CO₂ and 37° C. for 24 hr. After the reaction stopped, the cell was washed twice by PBS, scraped by scraper, put into micro tube, and centrifuged at more than 10,000 rpm for 5 min, and collected. After discarding the supernatant, the tube wherein only pellet is remained was added with lysis buffer and treated in ice for 5 min, and the cell was destroyed. After centrifuging it at more than 10,000 rpm for 5 min, the cell debris was left and only the supernatant was collected into a new tube. This Sample was stored at −70° C. until LTB₄ ELISA analysis.

Cell lysate was diluted in 1/20 with EIA buffer in the kit. LTB₄ standard was prepared to be 0, 0.04, 0.1, 0.2, 0.4, 1.0, 2.0, and 4.0 μg/ml. Leukotriene C₄ enzyme conjugate was prepared by diluting it in 1/50 with EIA buffer. 50 μl of the standard or the Sample and 50 μl of the diluted enzyme conjugate were put into antibody coating 96 well plate. After slow shaking, they were reacted at room temperature for 1 hr with covered.

After the reaction stopped, the plate was washed with 300 μl of wash buffer 3 times. 150 μl of Substrate was put into the plate and reacted for 30 min with slow shaking. The absorbance at 650 nm was measured, and the relative activity of Test groups compared with Control group was calculated in % by the following Calculation formula. Each value was standardized by Bradford protein quantity.

Calculation formula:
% of Inhibition=[(NC−PC)−(NC−S)/(NC−PC)]×100

\textcircled{3} Test result: 50% Inhibition activity against 5-LO of single extract and mixture (Unit: μg/ml)

| | 5-LO |
|---|---|
| Example 1 | 0.044 |
| Example 2 | 0.040 |
| Example 3 | 0.846 |
| Example 5 | 0.039 |
| Example 6 | 0.007 |
| Example 7 | 0.024 |
| Example 8 | 0.027 |

\textcircled{4} Conclusion: 5-LO inhibition activity of single extracts are in the order of Example 1=Example 2>>Example 3. And, mixtures wherein said single extracts were mixed in proper ratios showed 5-LO inhibition activity in the order of Example 6>Example 7≧Example 8>Example 5. Similar to the above test on COX-1, 2, in this test, Uncaria gambir crude extract also showed excellent 5-LO inhibition, whereas 5-LO inhibition activity of Scutellaria baicalensis extract was not significant. However, surprisingly, the mixture of said extracts showed synergistic 5-LO inhibition activity.

(3) Ear Swelling Inhibition Test (Swelling Inhibition Test)

\textcircled{1} Test Materials:

Test animals: ICR mouse (Daehan Bio Link)

Inflammation induction: Arachidonic acid (2 mg/20 μl)

Sample: Examples 1 to 8

Positive Control: Indomethacin (25, 50 mg/kg)

Concentration: 50, 75, 100 mg/kg

\textcircled{2} Test Methods: Experimental material was administered to the test animals (ICR mouse, Daehan Bio Link) 24 hr and 1 hr prior to inflammation production. Then, arachidonic acid was administered to the right ears of the test animals in a concentration of 2 mg/20 μl, acetone as control solvent was administered to the left ears, and the thickness of ears was measured by calipers to be used for solvent comparison. As control material, indomethacin, which is representative anti-inflammatory and antiphlogistic anodyne for NSAIDs, was administered orally 24 hr and 1 hr prior to administering arachidonic acid. The thickness of the test animal's ears whereto arachidonic acid and acetone were administered was measured at 1, 2 and 3 hr.

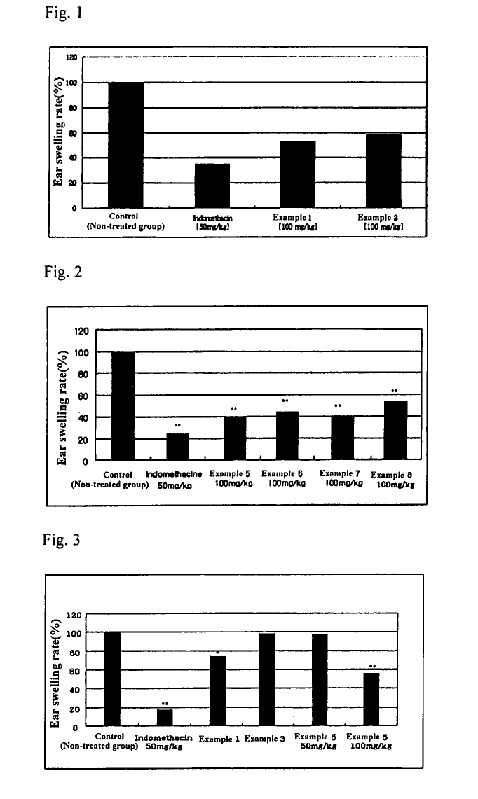

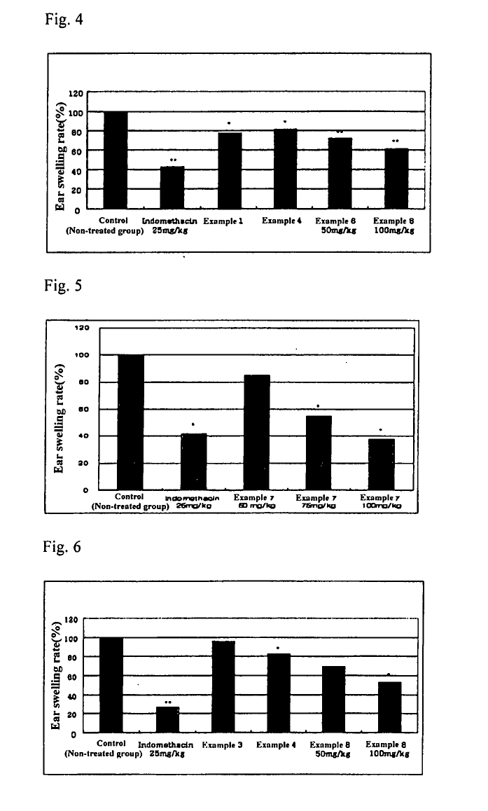

\textcircled{3} Conclusion: The anti-inflammatory activities of Examples 1 and 2 are shown in FIG. 1; the anti-inflammatory activities of Example 5 and Examples 1, 3 are shown in FIG. 3; the anti-inflammatory activities of Example 6 and Examples 1, 4 are shown in FIG. 4; the anti-inflammatory activities of Example 7 and Examples 1, 3, 4 are shown in FIG. 5; and the anti-inflammatory activities of Example 8 and Examples 1, 4 are shown in FIG. 6 (*P<0.05, **P<0.01).

As known from the above FIG. 1 to FIG. 6, Uncaria gambir single extract and its mixture showed the anti-inflammation effects. In particular, Example 1 showed stronger anti-inflammation effect than Example 3, and their mixture of Example 5 (100 mg/kg) showed synergistic effects from the mixing of single extracts. And, Example 5, which is the mixture of Examples 1 and 3, showed a concentration-dependent increasing tendency of anti-inflammatory activity.

As above, when Example 1 was mixed with Example 3 or Example 4, or Example 3 was mixed with Example 4, the composition (Examples 5 to 8) showed improved ear swelling inhibitory effects compared with Example 1 alone.

(4) Confirmation of Anti-Inflammatory Activity by CIA Model

\textcircled{1} Test Materials:

Test animals: DBA/1 mouse (Oriental Co., Ltd.)

Positive Control: Indomethacin (50 mg/kg)

Control Material: Glucosamine (250 mg/kg)

Sample: Example 5

Concentration: 100, 200 mg/kg

\textcircled{2} Test Methods: 8 week DBA/1 mice (Oriental Co., Ltd) were used to induce arthritis. To immunize the mice, 100 μg of collagen suspended with CFA (Complete Freund's Adjuvant) was administered to the mice's tails. After 21 days, arthritis was induced by 100 μg of collagen suspended with IFA (Incomplete Freund's Adjuvant). From the 21st day, the mice were divided into 5 groups, 5 mice in each group, and the prepared extract was administered to the mice until 56th day. Once a week, the weight, thickness of the paw having swelling, and point (0: normal, 1: slight redness, 2: swelling on toe, 3: severe swelling on overall, 4: most severe swelling on overall toe and joint) of the mice were measured. On 56th day, their blood was collected; autopsy was conducted on the mice; their paws having swelling were dyed by H & E (Hematozyline & Eosin) after the processes of fixing and decalcification; and the joint tissues were observed through microscope.

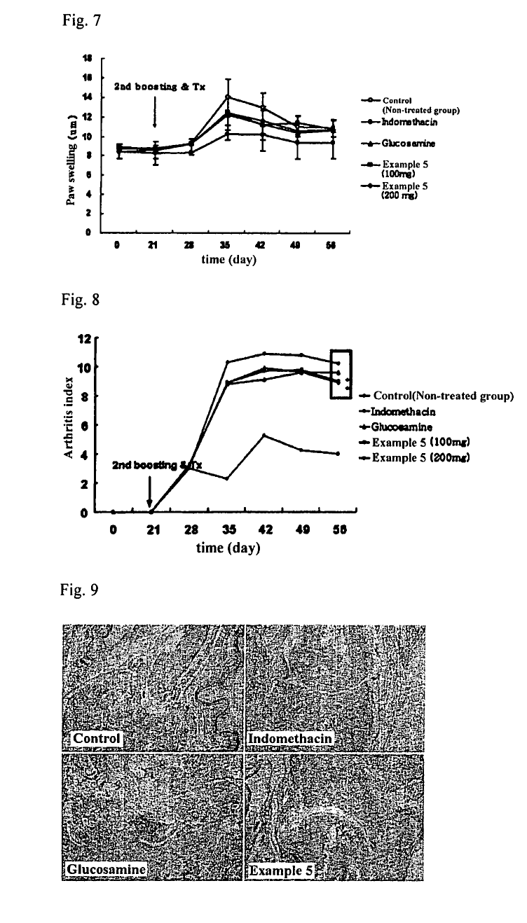

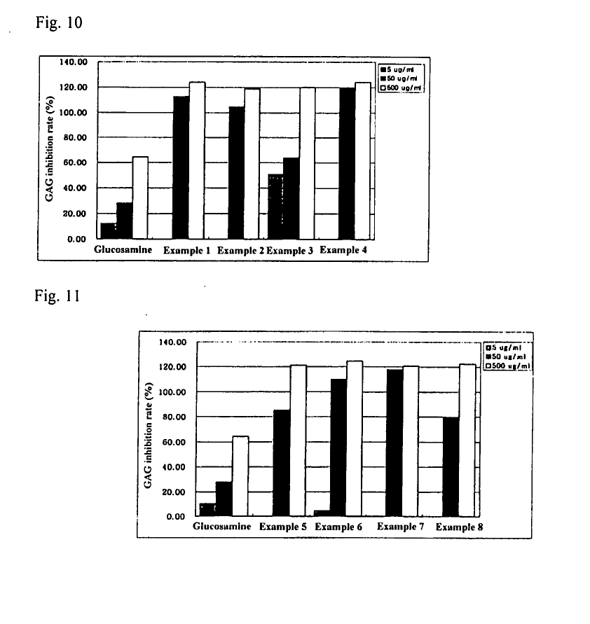

\textcircled{3} Conclusion: The change of swelling of the mouse paw with the passage of time and the change of arthritis index with the passage of time after administering collagen are shown in FIG. 7 and FIG. 8, respectively (*P<0.05). And, the cartilage tissue of CIA mouse joint after administering Example 5 is shown in FIG. 9.

As known from the above FIG. 7 to FIG. 9, the Example 5 administration group was about 20% effective compared with Control group. It was observed that the joint tissue's destruction and the immune cell's infiltration significantly decreased compared with Control group and the glucosamine administration group.

2) Joint Protection Test

(1) GAG analysis

\textcircled{1} Test Materials:

Materials: 6-well plate (Corning 3516), Dulbecco's modified Eagle's medium (DMEM, Biowhittaker), Heated inactivated fetal bovine serum (FBS), Penicillin-streptomycin (Gibco), IL-1 alpha (R&D 200LA-002), and Blyscan Glycosaminoglycan analysis kit (Biocolor B1000)

Sample: Examples 1 to 8

Control Material: Glucosamine

Concentration: 5, 50, 500 μg/ml

Test animal: New Zealand white rabbits (2.0 kg, 9 week) obtained from Samtako (Kyunggi-provence, Osan-si)

\textcircled{2} Test Methods

Cartilage explant cultures

Knee joint cartilage was collected from 9 week rabbit. Then, the collected cartilage was put into DMEM (5% FBS, penicillin 100U/ml, streptomycin 100 μg/ml) and stabilized in CO₂ culture medium of 37° C. for 24 hr. Before treating the sample, every cartilage was sliced by a certain size and put into 24 well. The prepared sample and IL-1 alpha (5 ng/ml) were treated. After reacting the treated sample in the 37° C., 5% CO₂ culture medium for 60 hr, the supernatant was collected and stored at −20° C., and used in the next test.

GAG analysis

To measure GAG secretion degree of the supernatant, Blyscan analysis kit was used. Absorbance at 656 nm was measured, and the relative activity of Test groups compared with Control group was calculated in % by the following Calculation formula.

Calculation formula:
Calculation formula=[(PC−Sample)−(PC−NC)]×100

NC: Negative-Control, PC: Positive-Control, S: Sample

\textcircled{3} Test result:

Joint protection effects of Examples 1 to 4 and 5 to 8 were shown in FIG. 10 and FIG. 11, respectively.

\textcircled{4} Conclusion: In particular, Examples 1 to 4 and 5 to 8 showed joint protection effects at ≦50 μg/ml concentration.

Formulation Example 1: Preparation of Solution

| | |
|---|---|
| Extract of Example 6 | 20 g |
| Sugar | 10 g |
| Isomerized sugar | 10 g |
| Smell of lemon | proper quantity |
| Total amount after adding purified water | 100 ml |

The above-mentioned ingredients were mixed according to a conventional preparation method for solution, and sterilized to give solution.

Formulation Example 2: Preparation of Solution

| | |
|---|---|
| Extract of Example 1 | 30 g |
| Sugar | 10 g |
| Isomerized sugar | 10 g |
| Smell of lemon | proper quantity |
| Total amount after adding purified water | 100 ml |

The above-mentioned ingredients were mixed according to a conventional preparation method for solution, and sterilized to give solution.

Formulation Example 3: Preparation of Capsule

| | |
|---|---|
| Extract of Example 7 | 500 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to a conventional preparation method for capsule to give capsule.

Formulation Example 4: Preparation of Capsule

| | |
|---|---|
| Extract of Example 8 | 500 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to a conventional preparation method for capsule to give capsule.

Formulation Example 5: Preparation of Capsule

| | |
|---|---|
| Extract of Example 5 | 500 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to a conventional preparation method for capsule to give capsule.

Formulation Example 6: Preparation of Capsule

| | |
|---|---|
| Extract of Example 1 | 700 mg |
| Lactose | 50 mg |
| Starch | 50 mg |
| Talc | 2 mg |
| Magnesium Stearate | proper quantity |

The above-mentioned ingredients were mixed, and filled in a gelatin capsule according to a conventional preparation method for capsule to give capsule.

Formulation Example 6: Preparation of Ointment

| | |
|---|---|
| Extract of Example 5 | 200 g |
| White Vaseline | 100 g |
| Stearyl alcohol | 150 g |
| Polyoxyethylene hydrogenated caster oil | 40 g |
| Glyceryl Monostearate | 20 g |
| Propylene glycol | 100 g |
| Methyl Parahydroxybenzoate | 1 g |
| Propyl Parahydroxygenzoate | 1 g |

The above-mentioned ingredients were mixed according to a conventional preparation method for ointment to give ointment.

### INDUSTRIAL APPLICABILITY

The present composition comprising Uncaria gambir extract showed excellent COX and/or 5-LO inhibition activity, the present composition additionally comprising Scutellaria baicalensis extract and/or Camellia sinensis extract showed synergistic effects, and the combined composition of Scutellaria baicalensis and Camellia sinensis extract also showed synergistic effects. The composition of the present invention is obtained from the natural material, and so shows excellent COX and/or 5-LO inhibition activity without danger of side effects, and thus can be used for the prevention or treatment of diseases including inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer. In addition, the present composition can be used for the prevention or treatment of various inflammatory diseases, including, in particular, osteoarthritis, rheumatoid arthritis, etc.

1. A composition for the prevention or treatment of physiological and pathological disorders mediated by cyclooxygenase (COX) and/or 5-lipoxygenase (5-LO) comprising Uncaria genus plant or its extract.

2. The composition according to claim 1, wherein the physiological and pathological disorders mediated by COX and/or 5-LO is selected from the group consisting of inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer.

3. The composition according to claim 2, wherein the physiological and pathological disorders mediated by COX and/or 5-LO is inflammatory disease.

4. The composition according to claim 1, wherein the Uncaria genus plant is selected from the group consisting of Uncaria gambir, U. attenuata Korth., U. borneensis Havil., U. callophylla Korth., U. elliptica R. Br., U. guianensis (Aubl.) Gmel., U. homomalla Miq., U. lanosa var. glabrata (B1.) Ridsd., U. macrophylla Wall., U. rhynchophylla Miq., U. sinensis (Oliv.) Havil., U tomentosa (Willd.) DC., U. yunnanensis Hsia K. C., U. hirsuta Havil., and U. lanosa var. appendiculata f. setiloba (Benth.) Ridsd.

5. The composition according to claim 4, wherein the Uncaria genus plant is Uncaria gambir.

6. The composition according to claim 1, additionally comprising Scutellaria baicalensis extract and/or Camellia sinensis extract.

7. The composition according to claim 6, wherein the extract is extracted with one or more polar solvent selected from the group consisting of water, acetone, or C₁₋₄ alcohol and isopropyl alcohol.

8. The composition according to claim 6, wherein the weight ratio of Uncaria gambir:Scutellaria baicalensis:Camellia sinensis is 0.1˜10:0.1˜10:0.1˜10.

9. A composition for the prevention or treatment of physiological and pathological disorders mediated by COX and/or 5-LO comprising Scutellaria baicalensis extract and Camellia sinensis extract.

10. The composition according to claim 9, wherein the physiological and pathological disorders mediated by COX and/or 5-LO is selected from the group consisting of inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer.

11. The composition according to claim 10, wherein the physiological and pathological disorders mediated by COX and/or 5-LO is inflammatory disease.

12. The composition according to claim 9, wherein the extract is extracted with one or more polar solvent selected from the group consisting of water, acetone, or C₁₋₄ alcohol and isopropyl alcohol.

13. The composition according to claim 9, wherein the weight ratio of Scutellaria baicalensis:Camellia sinensis is 0.1˜10:0.1˜10.

14. The composition according to claim 3, wherein the inflammatory disease is osteoarthritis or rheumatoid arthritis.

15. A use of Uncaria genus plant or its extract to prevent or treat physiological and pathological disorders mediated by COX and/or 5-LO.

16. The use according to claim 15, wherein the physiological and pathological disorders mediated by COX and/or 5-LO is selected from the group consisting of inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer.

17. The use according to claim 16, wherein the physiological and pathological disorder mediated by COX and/or 5-LO is inflammatory disease.

18. The use according to claim 15, wherein the Uncaria genus plant is selected from the group consisting of Uncaria gambir, U. attenuata Korth., U. borneensis Havil., U. callophylla Korth., U. elliptica R. Br., U. guianensis (Aubl.) Gmel., U. homomalla Miq., U. lanosa var. glabrata (B1.) Ridsd., U. macrophylla Wall., U. rhynchophylla Miq., U. sinensis (Oliv.) Havil., U. tomentosa (Willd.) DC., U. yunnanensis Hsia K. C., U. hirsuta Havil., and U. lanosa var. appendiculata f. setiloba (Benth.) Ridsd.

19. The use according to claim 18, wherein the Uncaria genus plant is Uncaria gambir.

20. A use of a composition comprising Scutellaria baicalensis extract and Camellia sinensis extract to prevent or treat a physiological and pathological disorders mediated by COX and/or 5-LO.

21. The use according to claim 20, wherein the physiological and pathological disorder mediated by COX and/or 5-LO is selected from the group consisting of inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer.

22. The use according to claim 21, wherein the physiological and pathological disorder mediated by COX and/or 5-LO is inflammatory disease.

23. The use according to claim 20, wherein the extract is extracted with one or more polar solvent selected from the group consisting of water, acetone, or C₁₋₄ alcohol and isopropyl alcohol.

24. The use according to claim 20, wherein the weight ratio of Scutellaria baicalensis:Camellia sinensis is 0.1˜10:0.1˜10.

25. The use according to claim 17, wherein the inflammatory disease is osteoarthritis or rheumatoid arthritis.

26. A use of a composition for the prevention or treatment of physiological and pathological disorders mediated by COX and/or 5-LO, comprising, i) Uncaria genus plant or its extract, and ii) Scutellaria baicalensis extract and/or Camellia sinensis extract.

27. The use according to claim 26, wherein the Uncaria genus plant is Uncaria gambir.

28. The use according to claim 26, wherein the weight ratio of Uncaria gambir:Scutellaria baicalensis:Camellia sinensis is 0.1˜10:0.1˜10:0.1˜10.

29. A method for preventing or treating physiological and pathological disorders mediated by COX and/or 5-LO, comprising administering a therapeutically effective amount of a composition comprising Uncaria genus plant or its extract to mammal.

30. The method according to claim 29, wherein the physiological and pathological disorder mediated by COX and/or 5-LO is selected from the group consisting of inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer.

31. The method according to claim 30, wherein the physiological and pathological disorders mediated by COX and/or 5-LO is inflammatory disease.

32. The method according to claim 29, wherein the Uncaria genus plant is selected from the group consisting of Uncaria gambir, U. attenuata Korth., U. borneensis Havil., U. callophylla Korth., U. elliptica R. Br., U. guianensis (Aubl.) Gmel., U. homomalla Miq., U. lanosa var. glabrata (B1.) Ridsd., U. macrophylla Wall., U. rhynchophylla Miq., U. sinensis (Oliv.) Havil., U. tomentosa (Willd.) DC., U. yunnanensis Hsia K. C., U. hirsuta Havil., and U. lanosa var. appendiculata f. setiloba (Benth.) Ridsd.

33. The method according to claim 32, wherein the Uncaria genus plant is Uncaria gambir.

34. The method according to claim 29, wherein the composition additionally comprises Scutellaria baicalensis extract and/or Camellia sinensis extract.

35. The method according to claim 34, wherein the extract is extracted with one or more polar solvent selected from the group consisting of water, acetone, or C₁₋₄ alcohol and isopropyl alcohol.

36. The method according to claim 34, wherein the weight ratio of Uncaria gambir:Scutellaria baicalensis:Camellia sinensis is 0.1˜10:0.1˜10:0.1˜10.

37. A method for preventing or treating physiological and pathological disorders mediated by COX and/or 5-LO, comprising administering a therapeutically effective amount of a composition comprising Scutellaria baicalensis and Camellia sinensis extract to mammal.

38. The method according to claim 37, wherein the physiological and pathological disorders mediated by COX and/or 5-LO is selected from the group consisting of inflammatory disease, menstrual pain, arteriosclerosis, heart attack, obesity, diabetes, X syndromes, decrease of cognitive function, Alzheimer's disease, respiratory allergic reaction, chronic venous insufficiency, hemorrhoids, systemic lupous erythematosus, psoriasis, chronic tension headache, migraine, inflammatory enteropathy, local infectious disease by virus, bacteria and germ, sunburn, burn, contagious dermatitis, melanoma and cancer.

39. The method according to claim 38, wherein the physiological and pathological disorder mediated by COX and/or 5-LO is inflammatory disease.

40. The method according to claim 37, wherein the extract is extracted with one or more polar solvent selected from the group consisting of water, acetone, or C₁₋₄ alcohol and isopropyl alcohol.

41. The method according to claim 37, wherein the weight ratio of Scutellaria baicalensis:Camellia sinensis is 0.1˜10:0.1˜10.

42. The method according to claim 31, wherein the inflammatory disease is osteoarthritis or rheumatoid arthritis.

43. A method of preparing an agent for the prevention or treatment of physiological and pathological disorders mediated by COX and/or 5-LO by mixing Uncaria genus plant or its extract with Scutellaria baicalensis extract and/or Camellia sinensis extract in the weight ratio of 0.1˜10:0.1˜10.

44. The method according to claim 43, wherein the Uncaria gambir, Scutellaria baicalensis and Camellia sinensis are mixed in the weight ratio of 0.1˜10:0.1˜10:0.1˜10.

45. A method of preparing an agent for the prevention or treatment of physiological and pathological disorders mediated by COX and/or 5-LO by mixing Scutellaria baicalensis extract with Camellia sinensis extract in the weight ratio of 0.1˜10:0.1˜10.
